(19) United States
(12) Patent Application Publication
(10) Pub. No.: US 2015/0080580 A1
(43) Pub. Date: Mar. 19, 2015

(54) THIOUREA DERIVATIVES AS a-CHYMOTRYPSIN INHIBITORS

(71) Applicants: Atta-ur Rahman, Karachi (PK); Bishnu P. Marasini, Karachi (PK); Muhammad Iqbal Choudhary, Karachi (PK); Khalid M. Khan, Karachi (PK); Farzana Sheikh, Karachi (PK); Atia-tul- Wahab, Karachi (PK)

(72) Inventors: Atta-ur Rahman, Karachi (PK); Bishnu P. Marasini, Karachi (PK); Muhammad Iqbal Choudhary, Karachi (PK); Khalid M. Khan, Karachi (PK); Farzana Sheikh, Karachi (PK); Atia-tul- Wahab, Karachi (PK)

(21) Appl. No.: 14/030,817

(22) Filed: Sep. 18, 2013

(51) Int. Cl. C07D 213/75; C07C 335/18; C07D 213/59; C07C 335/16

(52) U.S. Cl. CPC C07D 213/75; C07C 335/16; C07C 335/18; C07D 213/59
USPC 546/305; 564/29; 546/331

(57) ABSTRACT

α-Chymotrypsin inhibitors of thiourea class are reported that could be potent inhibitors of proteases, elastases, proteasomes, NS3 and NS4 serine protease of hepatitis C virus, dengue virus, etc. Compounds 1-22, which are belong to thiourea class, showed good inhibition. Their kinetics study and cytotoxicity profiles showed all type of inhibition except uncompetitive-type inhibition and no cytotoxicity except few compounds. Competitive type of inhibitors could inhibit other α-chymotrypsin-like serine proteases, which are therapeutics target.

### BACKGROUND OF THE INVENTION

Serine proteases are responsible for many functions in our body, such as, blood clotting, in digestion of protein in food intake and activation of complement systems, etc. However, in abnormal conditions, serine proteases are also responsible for destruction of the body's own cellular proteins and peptides such as in thrombosis, inflammation, diabetes, chronic pancreatitis, etc. (Wilcox, 1970; Turk, 2006; Bachovchin and Cravatt, 2012; Rosendahl et. al., 2008). Similarly, viral serine proteases help in replication of some viruses. The NS3 or NS4 (non-structural protein-3, -4) of viruses of hepatitis C, West Nile, dengue, etc. is an essential component for virus maturation. Hence, serine proteases are the ideal target for the development of drugs for viral infections (Ekonomiuk et. al., 2009; Chanprapaph et. al., 2005; Wyles et. al., 2008).

α-Chymotrypsin (EC No. 3.4.21.1), a serine protease containing Asp^{102,} His⁵⁷ and Ser¹⁹⁵ in catalytic triad, catalyzes the breakdown of polypeptide and proteins. It is secreted by pancreas as exocrine, and biosynthesized by the acinar cells of pancreas as inactive zymogen, chymotrypsinogen so that it does not digest the pancreas. After release of inactive chymotrypsinogen in the small intestine, it is activated upon proteolytic cleavage by trypsin or enterokinase and enhances to digest protein content in our food intake. The mucus layer in the intestine prevents digestion of body's own tissue by α-chymotrypsin. If the inactive chymotrypsin, the chymotrypsinogen, is cleaved to become active enzyme, then it may digest body's own proteins or tissues, such as in cases of pancreatitis (Zhou and Toth, 2011; Wilcox, 1970). Epithelial sodium channel (EnaC) is activated by α-chymotrypsin via proteolytic cleavage. This activated EnaC is one of the major factors to initiate cystic fibrosis (Rauh et al., 2010).

α-Chymotrypsin and cathepsin G together cleave interleukin 1β (IL-1β) precursor into functional and active IL-1β which initiates arthritis cascade (Stehlik, 2009). α-Chymotrypsin shares the structure and functional similarity with other chymotrypsin-like serine proteases, such as elastase, 20S proteosome, NS3 and NS4 serine protease of hepatitis C virus, dengue virus, etc. Interestingly, these inhibitors have the possibility to inhibit more than one type of serine protease (Zollner, 1989). α-Chymotrypsin can be targeted as the preliminary step in drug development against various protease and against many physiological abnormalities.

Thiourea derivatives have been found to be inhibitors of many serine proteases, such as factor Xa, plasmin, NS4A protein of hepatitis C virus (Bisacchi et al., 2005; Wyles et al., 2008). Furthermore, thiourea derivatives have shown inhibition against various leukemias and solid tumors (Li et al., 2009) melanogenesis and tyrosinase enzyme (Thanigaimalai et al., 2011). Thiourea also has been found to facilitate the transport of anions across lipid bilayers, a remedy for “channelopathies” (Busschaert et al., 2012). Some thiourea derivatives have been used as medicine also, e.g., propylthiouracil, which is used to treat hyperthyroidism, Graves' disease, etc. (Nakamura et al., 2007); zevalin (ibritumomab tiuxetan) is used for the treatment of B cell non-Hodgkin's lymphoma (Arrichiello et al., 2012); thiocarlide is used in the treatment of tuberculosis, (Phetsuksiri et al., 2003). This indicates that new thiourea derivatives might show efficacy as potent therapeutics against the diseases related to α-chymotrypsin and other serine proteases.

### BRIEF SUMMARY OF THE INVENTION

Serine proteases are the target for the development of drugs for many pathological conditions, such as thrombosis, pancreatitis, diabetes, hepatitis C, dengue, malaria, etc. α-Chymotrypsin (EC 3.4.21.1), a serine protease, is secreted by pancreas as inactive zymogen. Premature activation of α-chymotrypsin increases the chance of digestion of body's own tissues. Most of the reported serine and α-chymotrypsin inhibitors are peptide in nature. We are reporting non-peptide molecules of the class thiourea as α-chymotrypsin inhibitors. Among 35 molecules, 22 compounds showed inhibition with IC₅₀ values ranged from 11.6 to 386.9 μM. Only 4 (compound nos. 5, 7, 8 and 9) of these 22 inhibitors showed growth inhibition for 3T3 cell lines. The others were not cytotoxic. Hence, these molecules could be potent therapeutics for pancreatitis as α-chymotrypsin inhibitors as well as other diseases due to serine proteases, such as thrombosis, hepatitis C, dengue, etc.

### BRIEF DESCRIPTION OF THE DRAWING

FIG. 1 depicts (A) Line-weaver Burk plot of different concentrations of compound 1 as mentioned in the inset legend. In this steady state kinetics, all the lines meet at 2^{nd} quadrant indicates mixed-type of inhibition. (B) Secondary plot produced from Line-weaver Burk plot. The meeting point of the straight line at x-axis is equivalent to Ki value. (C) Dixon plot of compound 1 with different concentration of substrate as mentioned in the inset legend. In this graph, all the lines meet at 2^{nd} quadrant indicates mixed-type of inhibition, and x-axis value from the meeting point of these straight lines is equivalent to Ki value. (D) Secondary plot produced from Dixon plot for compound 1. The line does not meet at origin clarifies confusion with competitive-type of inhibition. (E) Hanes-Woolf plot of various concentrations of compound 1 as mentioned in the inset legend. These straight lines meet at 3^{rd} quadrant further confirms as this compound showed mixed-type of inhibition.

FIG. 2 depicts (A) Line-weaver Burk plot of compound 2, where all lines met at x-axis indicating non-competitive-type of inhibition, and (B) Secondary plot produced from Line-weaver Burk plot. The meeting point of the straight line at x-axis is equivalent to Ki value of compound 2. (C) Dixon plot of compound 2 with different concentration of substrate as shown in the inset. The meeting point of lines at x-axis not only indicated as non-competitive type but also Ki value. (D) Secondary plot from Dixon plot for compound 2. (E) Hanes-Woolf plot of various concentrations of compound 2 as mentioned in the inset legend. The lines meeting at the x-axis in the figure further validates as non-competitive-type of inhibition by compound 2.

FIG. 3 depicts (A) Line-weaver Burk plot of different concentrations of compound 20 as mentioned in the inset legend. In this steady state kinetics, all the lines meet at y-axis indicates competitive-type of inhibition. (B) Secondary plot produced from Line-weaver Burk plot. The meeting point of the straight line at x-axis is equivalent to Ki value. (C) Dixon plot of compound 20 with different concentration of substrate as mentioned in the inset legend. In this graph, these entire lines meet at 2^{nd} quadrant indicates competitive type of inhibition, the meeting point of these straight lines at x-axis is equivalent to Ki value. (D) Secondary plot produced from Dixon plot for compound 20. The line passes through origin clarifies confusion with mixed-type of inhibition. (E) Hanes-Woolf plot of various concentrations of compound 20 as mentioned in the inset legend. These straight lines parallel to each other confirms as this compound is competitive-type of inhibitor.

FIG. 4 depicts (A) Line-weaver Burk plot (B) Secondary plot produced from Line-weaver Burk plot. The meeting point of the straight line at x-axis is equivalent to Ki value. (C) Dixon plot of compound 3. All the legends are shown in figure.

FIG. 5 depicts (A) Line-weaver Burk plot (B) Secondary plot produced from Line-weaver Burk plot. The meeting point of the straight line at x-axis is equivalent to Ki value. (C) Dixon plot of compound 4. All the legends are shown in figure.

FIG. 6 depicts (A) Line-weaver Burk plot (B) Secondary plot produced from Line-weaver Burk plot. The meeting point of the straight line at x-axis is equivalent to Ki value. (C) Dixon plot of compound 5. All the legends are shown in figure.

FIG. 7 depicts (A) Line-weaver Burk plot (B) Secondary plot produced from Line-weaver Burk plot. The meeting point of the straight line at x-axis is equivalent to Ki value. (C) Dixon plot of compound 6. All the legends are shown in figure.

FIG. 8 depicts (A) Line-weaver Burk plot (B) Secondary plot produced from Line-weaver Burk plot. The meeting point of the straight line at x-axis is equivalent to Ki value. (C) Dixon plot of compound 7. All the legends are shown in figure.

FIG. 9 depicts (A) Line-weaver Burk plot (B) Secondary plot produced from Line-weaver Burk plot. The meeting point of the straight line at x-axis is equivalent to Ki value. (C) Dixon plot of compound 8. All the legends are shown in figure.

FIG. 10 depicts (A) Line-weaver Burk plot (B) Secondary plot produced from Line-weaver Burk plot. The meeting point of the straight line at x-axis is equivalent to Ki value. (C) Dixon plot of compound 9. All the legends are shown in figure.

FIG. 11 depicts (A) Line-weaver Burk plot (B) Secondary plot produced from Line-weaver Burk plot. The meeting point of the straight line at x-axis is equivalent to Ki value. (C) Dixon plot of compound 10. All the legends are shown in figure.

FIG. 12 depicts (A) Line-weaver Burk plot (B) Secondary plot produced from Line-weaver Burk plot. The meeting point of the straight line at x-axis is equivalent to Ki value. (C) Dixon plot of compound 11. All the legends are shown in figure.

FIG. 13 depicts (A) Line-weaver Burk plot (B) Secondary plot produced from Line-weaver Burk plot. The meeting point of the straight line at x-axis is equivalent to Ki value. (C) Dixon plot of compound 12. All the legends are shown in figure.

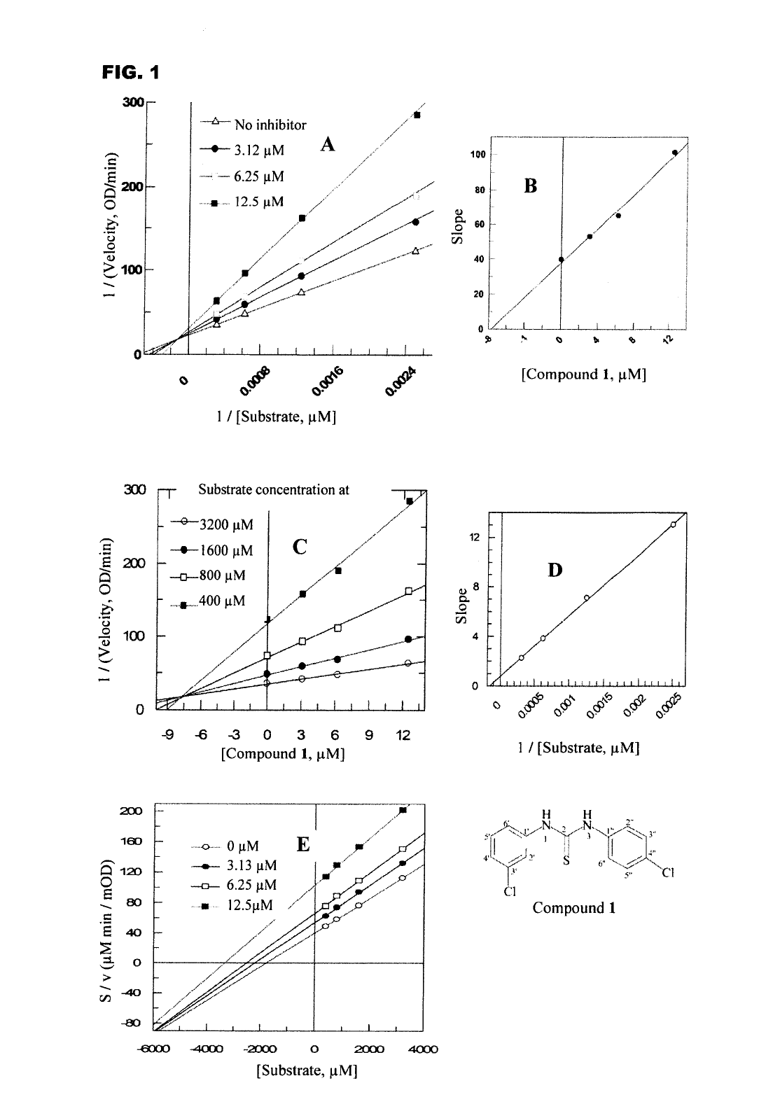

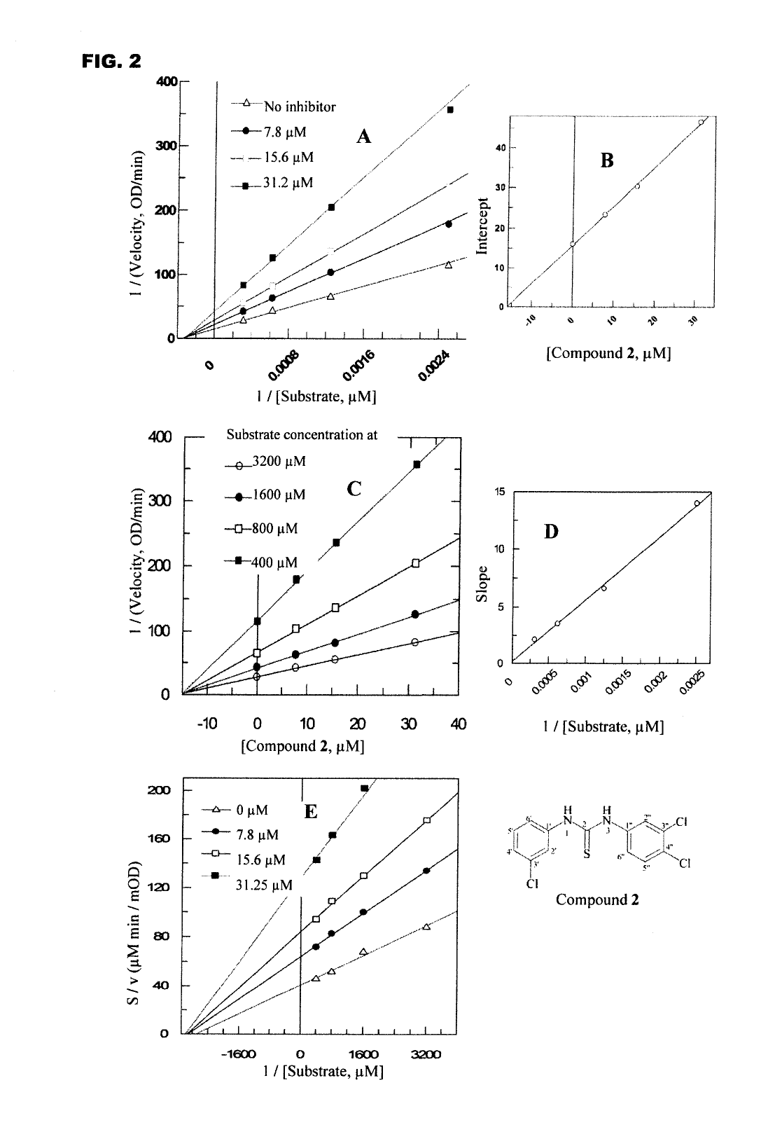

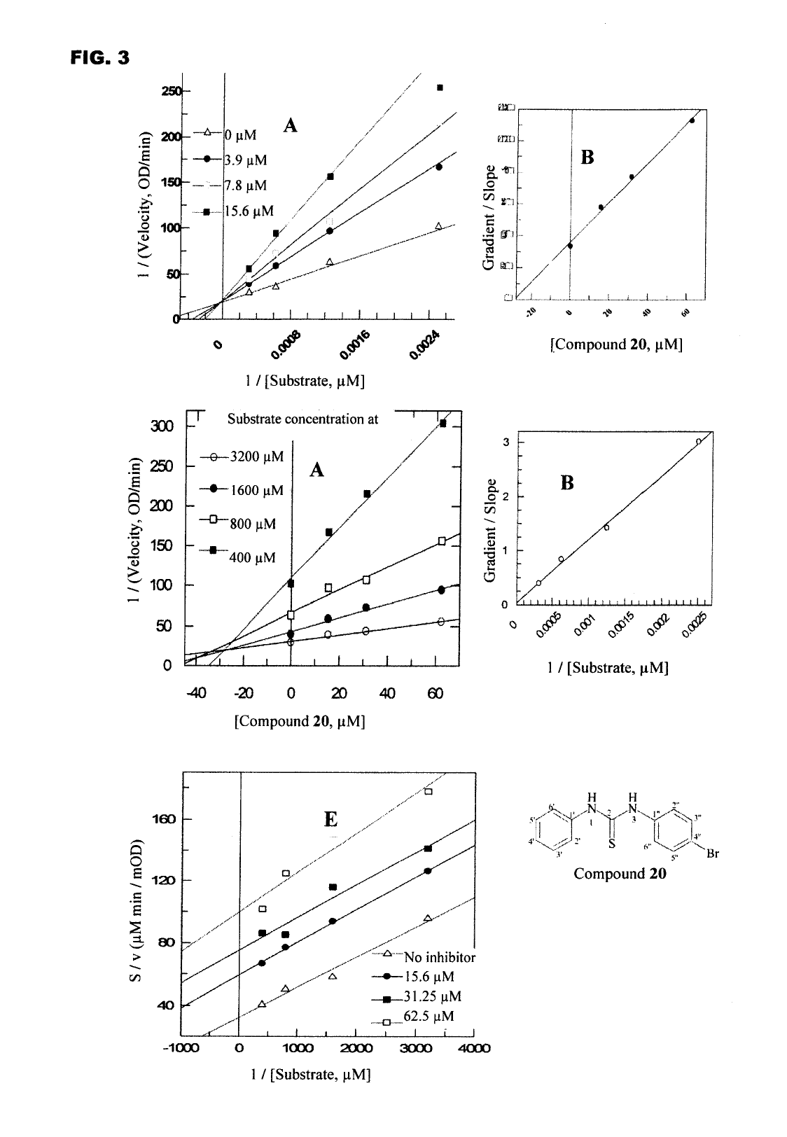

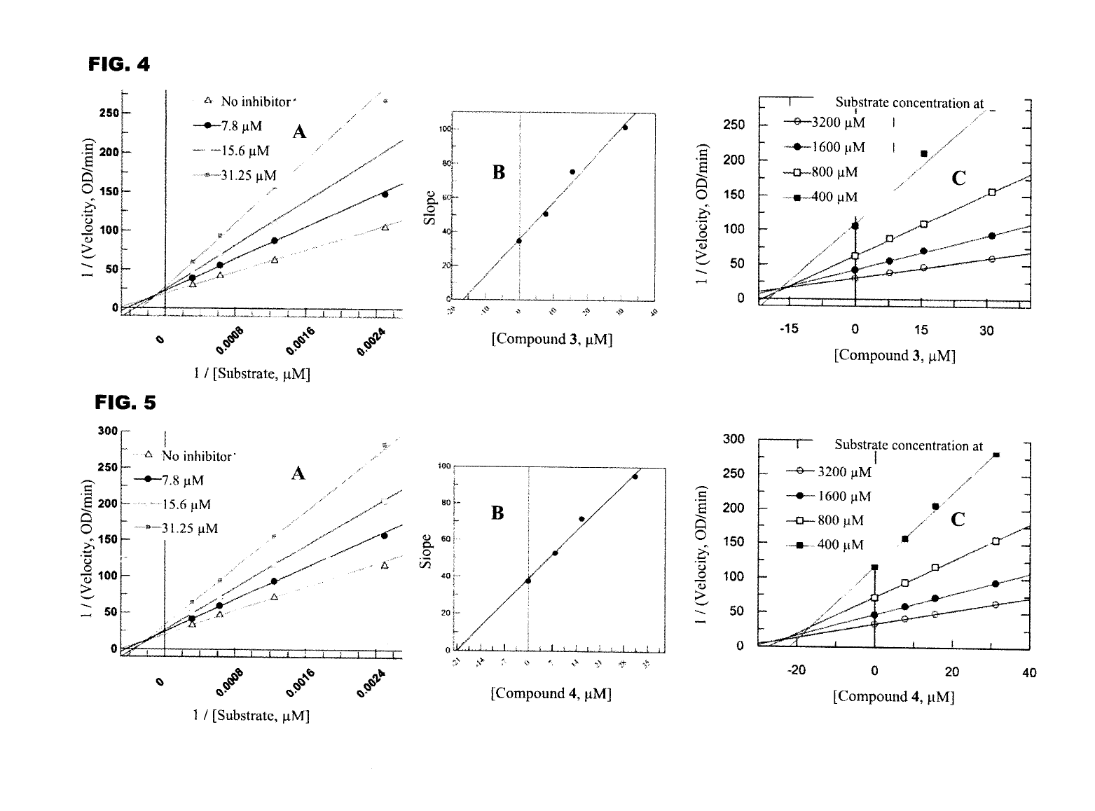

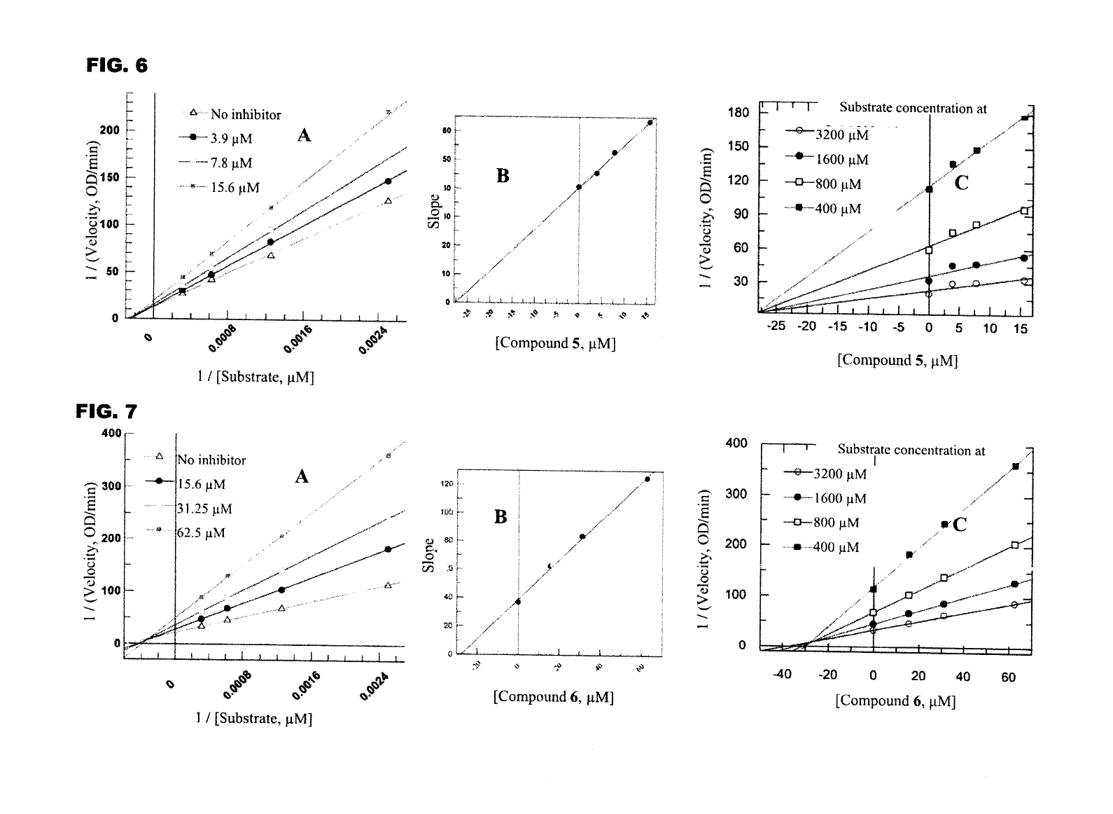

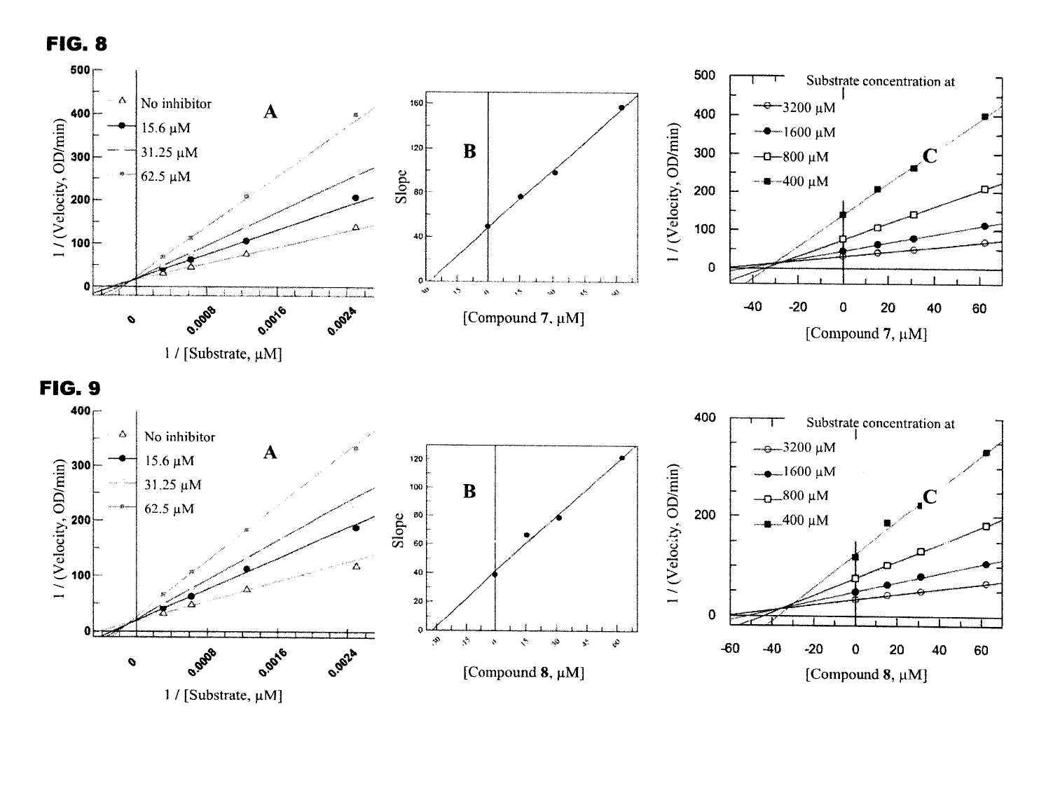

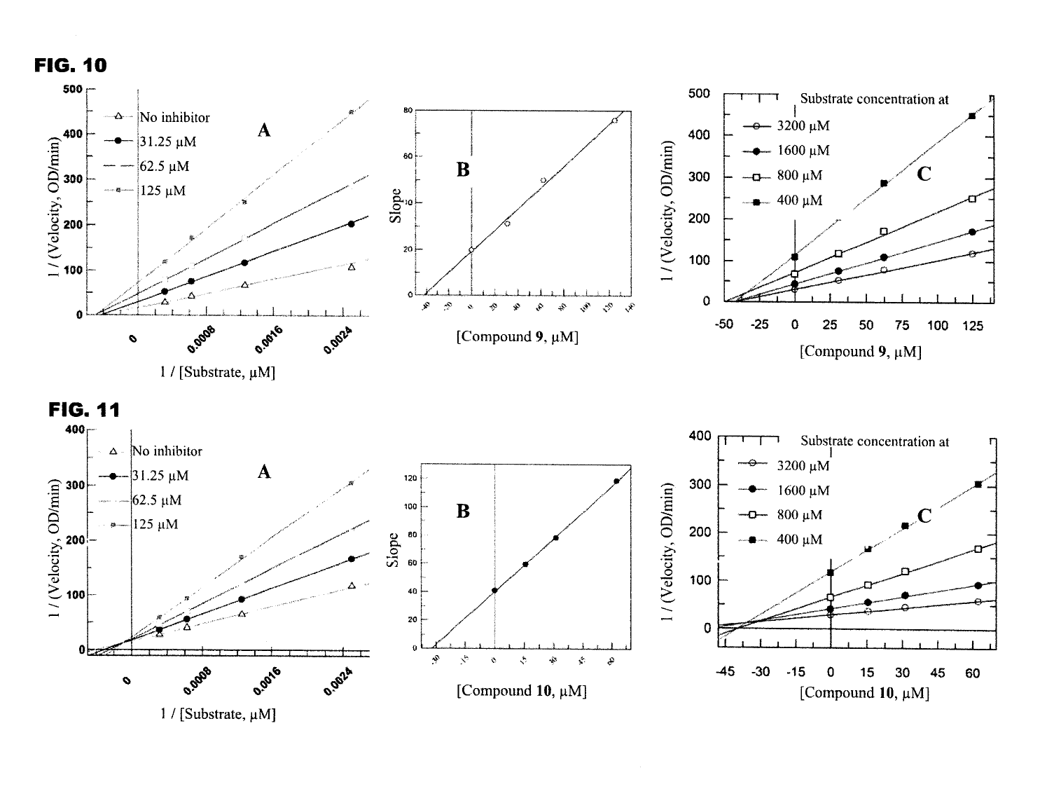

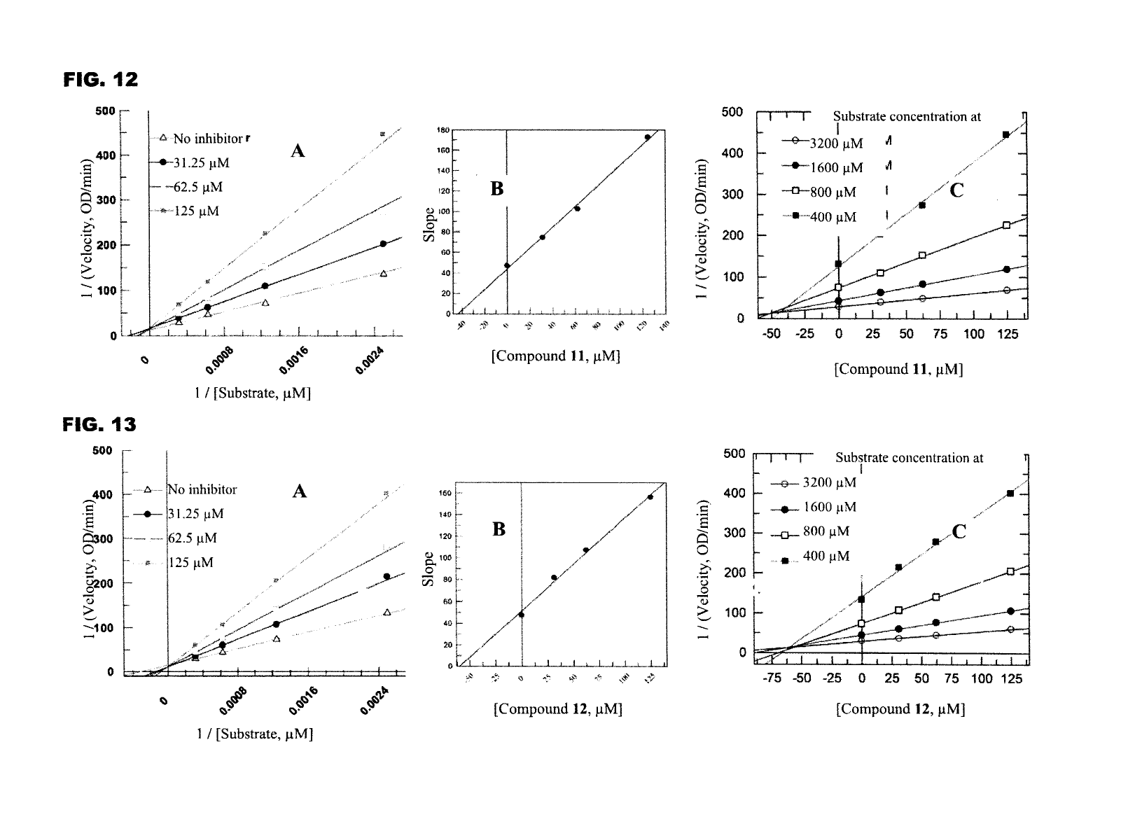

### DETAILED DESCRIPTION OF THE INVENTION

The present invention relates to discovery and mechanism of inhibition of the molecules that inhibit α-chymotrypsin enzyme.

α-Chymotrypsin (bovine pancreas, C4129) and N-succinyl-L-phenylalanine-p-nitroaniline (S2628) were purchased from Sigma-Aldrich (USA). Chymostatin (152845) from MP Biomedicals, LLC, USA, Tris(hydroxymethyl)aminomethane from Scharlau Chemie Barcelona, Spain and all other reagents were obtained from E. Merck and were of analytical grade. All other solvents and reagents were of analytical grade and used directly without purification.

This was performed in 50 mM Tris-HCl buffer pH 7.6 with 10 mM CaCl₂. α-Chymotrypsin (12 units/mL prepared in 50 mM Tris-HCl buffer) with the different concentration of test compounds prepared in DMSO. It was then incubated at 30° C. for 25 min. The reaction was started by the addition of the substrate, N-succinyl-L-phenylalanine-p-nitroanilide (final concentration of 0.4 mM, prepared in the buffer as mentioned above). The change in absorbance due to released p-nitroaniline was continuously monitored at 410 nm (Choudhary et al., 2011). All the experiments were in a final volume of 200 μL in triplicate, and data was taken using a micro-plate reader (SpectraMax M2, Molecular Devices, Calif., USA).

The results were processed by using SoftMax Pro 4.8 then analyzed by MS Excel software. The % of inhibition based upon initial velocity and calculated as:

$$\% inhibition = 100 - ( \frac{OD / min of test compound}{OD / min of positive control} ) \times 100$$

IC₅₀ (Inhibition of enzymatic hydrolysis of the substrate SPpNA by 50%) value was calculated using EZ-Fit software (Perellela Scientific, Inc., Amherst, Mars, USA).

### α-Chymotrypsin Inhibition Kinetic Study

The change in concentration of the substrate/product (rate of reaction) was measured as optical density per minute (OD/min). This OD/min was obtained by incorporating different concentrations of compounds over substrate (SPpNA) concentrations between 0.4 mM and 3.2 mM in micro-plate reader. Reciprocal of OD/min against the reciprocal of the substrate concentration, defined as Lineweaver-Burk plot (and its secondary plot; slope vs compound concentration); the Dixon plot (and its secondary plot; slope vs reciprocal of compound concentration) and then Hanes-Woolf plot were plotted (Lineweaver et al., 1934; Dixon, 1953; Segel, 1993) Graphs were plotted using GraFit 4 (Erithacus Software Limited, Surrey, UK) and GraphPad Prism 5 (GraphPad Software, California, USA). The types of inhibition were determined by the graphical views of Dixon plots, Lineweaver-Burk plots and their secondary plots as well as Hanes-Woolf plot. The Ki values, obtained directly from the software, also were cross-checked and tallied in these graphs. Final concentration of DMSO was maintained 5.5%.

### Cell Proliferation Inhibition (Cytotoxicity) Assay for 3T3 Cell Line

It was evaluated by using the MTT (3-[4,5-dimethylthiazole-2-yl]-2,5-diphenyl-tetrazolium bromide) assay (Mosmann, 1983). The 3T3 cell-lines (Mouse Fibroblasts) were cultured in Dulbecco's Modified Eagle's Medium, which was later supplemented with 5% of bovine serum, 100 IU/mL of penicillin, and 100 μg/mL of streptomycin, in 5% CO₂ incubator at 37° C. Exponentially growing cell, 1×10⁴ cells/well incorporated into each well of 96-well plates. Then, fresh the medium containing different concentrations of compounds were loaded in each well after 24 hours. Again, after 72 hrs, fresh the medium containing 0.5 mg/mL MTT were loaded in each well, and incubated further for 4 hrs. Subsequently, the media containing MTT was replaced by 100 μL of DMSO for 15 min, and then the absorbance was taken at 570 nm.

Thiourea had shown enzymatic inhibition of some serine proteases as well as they are used as medicine, such as propylthiouracil, zevalin (ibritumomab tiuxetan), thiocarlide. Considering the therapeutic significance of the thiourea as mentioned above, compounds 1-35 were synthesized and evaluated for their α-chymotrypsin inhibitory potential. Results are presented in Table 1. Out of thirty five compounds, twenty three compounds showed a moderate to good inhibitory effect.

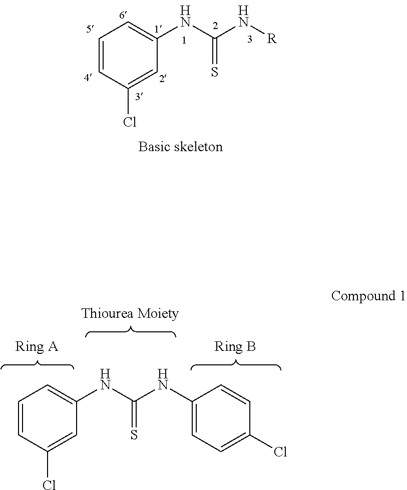

Compound 1 (IC₅₀=11.6 μM) with a chloro substituent at para-position of ring B found to be more potent than 3 (IC₅₀=23.3 μM) where chloro substituent lies at meta-position of ring B. Comparison based upon bis-chlorine substituent on ring B; potency of the compounds were found in the order of 2>8>13>16. In compound 1, chlorine is in para-position, whereas in 3, chlorine is in meta-position, while in compound 23, which failed to show any inhibition, chlorine is in ortho-position. In compound 2, two chlorine are attached to meta- and para-position; in 8, ortho- and para-position; in 3, ortho- and meta-position; in 6, ortho-1- and ortho-2-position. In all of these cases, more inhibitory activity exhibited if the chlorine substituent remains farthest from the core thiourea moiety.

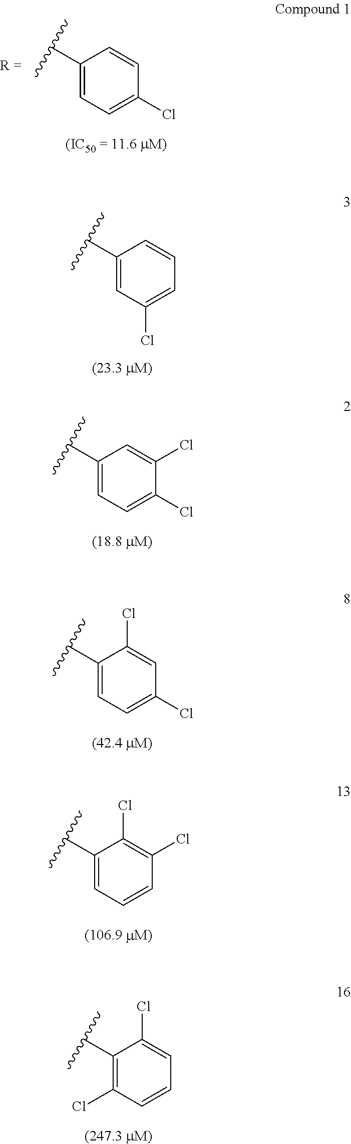 [SMILES: *c1ccc(Cl)cc1 ; *c1cccc(Cl)c1 ; *c1ccc(Cl)c(Cl)c1 ; *c1ccc(Cl)cc1Cl ; *c1cccc(Cl)c1Cl ; *c1c(Cl)cccc1Cl]

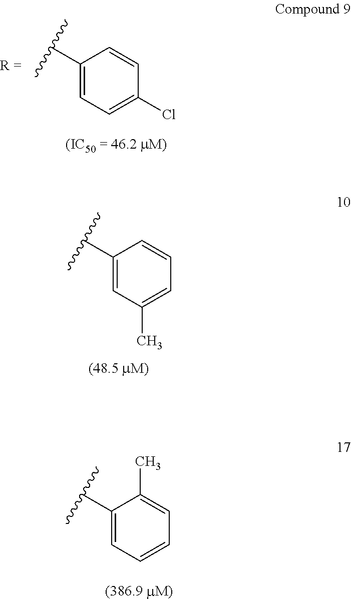

Based on —CH₃ substituent on ring B; potency of these compounds were found to be in the order of 9>10>17. In compound 9, —CH₃ is in para position whereas in 10, —CH₃ is in meta-position; and in 36, —CH₃ is in ortho-position. In all these cases, the farther —CH₃ from the core thiourea, more inhibitory activity was observed. Similar result found for —Br substituent i.e., compound 10 found to be more potent than 20. In case of electron donating group, such as, methoxy group, there is no significant difference in the inhibition effect by their position in ring B. Compound 4, 5 and 6 showed almost same inhibition potency, where methoxy group are in meta-, ortho- and para-position, respectively. But two methoxy groups attached in the same ring found to be responsible to decrease activity of the thiourea derivate as in compound 27.

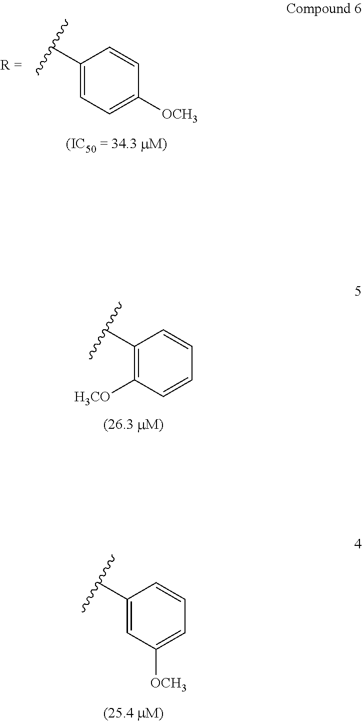 [SMILES: *c1ccc(OC)cc1 ; *c1ccccc1OC ; *c1cccc(OC)c1]

All of the above mentioned cases indicate the sulphur or amines moiety of thiourea may interact with the enzyme. These electron withdrawing groups may create steric hindrance or make the electron of sulphur or amines moiety unavailable to make hydrogen bond with the enzyme. However, the core thiourea moiety (H₂NCSNH₂) alone failed to show any significant inhibition. It probably needs at least one benzene ring to be fitted in oxyanion hole or other binding pocket (Wallace et al., 1963; Neurath et al., 1951).

Compounds 19 and 26 contain pyridine moiety instead of ring B. The chloro substituent at C-2 of pyridine ring in compound 26 is responsible of decreased potency of the compound. Similarly, compound 29 is inactive due to lack of chloro substituent in ring A, as compared to compound 19 (IC₅₀=199.3 μM). Benzene ring with chloro substitution might increase the inhibition potency by enabling a better fit in the oxyanion hole of the α-chymotrypsin enzyme. The position at which pyridine-substituted ring is attached on thiourea core does not play any role in the inhibition.

TABLE 2
Inhibition of α-chymotrypsin by thiourea derivatives 1-35 and their cytotoxicity for
3T3 cell line.

| | | | | | | 3T3 cell-line |
|---|---|---|---|---|---|---|
| | | | IC₅₀ ± SEM | Ki ± SEM | Type of | GI₅₀ ± SEM |
| Compound | Structures | IUPAC Name | (μM) | (μM) | inhibition | (μM) |
| 1 | 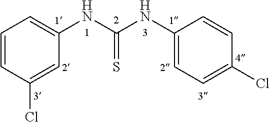 [SMILES: S=C(Nc1ccc(Cl)cc1)Nc1cccc(Cl)c1] | 1-(3-Chlorophenyl)- 3-(4-chlorophenyl) thiourea | 11.6 ± 0.1 | 8.6 ± 0.8 | Mixed | >30 |
| 2 | 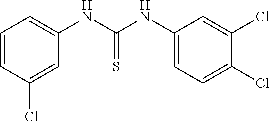 [SMILES: S=C(Nc1cccc(Cl)c1)Nc1ccc(Cl)c(Cl)c1] | 1-(3-Chlorophenyl)- 3-(3,4- dichlorophenyl) thiourea | 18.8 ± 0.2 | 15.5 ± 0.4 | Non- Competitive | >30 |
| 3 | 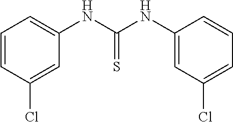 [SMILES: S=C(Nc1cccc(Cl)c1)Nc1cccc(Cl)c1] | 1,3-Bis(3- chlorophenyl) thiourea | 23.3 ± 0.6 | 15.9 ± 1.5 | Mixed | >30 |
| 4 | 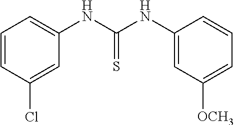 [SMILES: COc1cccc(NC(=S)Nc2cccc(Cl)c2)c1] | 1-(3-Chlorophenyl)- 3-(3-methoxyphenyl) thiourea | 25.4 ± 0.2 | 21.2 ± 1.5 | Mixed | >30 |
| 5 | 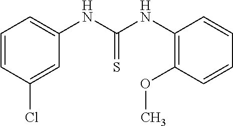 [SMILES: COc1ccccc1NC(=S)Nc1cccc(Cl)c1] | 1-(3-Chlorophenyl)- 3-(2-methoxyphenyl) thiourea | 26.3 ± 4.9 | 25.3 ± 4.6 | Non- Competitive | 10.7 ± 1.2 |
| 6 | 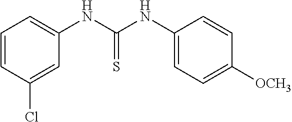 [SMILES: COc1ccc(NC(=S)Nc2cccc(Cl)c2)cc1] | 1-(3-Chlorophenyl)- 3-(4-methoxyphenyl) thiourea | 34.3 ± 2.8 | 25.6 ± 0.9 | Mixed | >30 |
| 7 | 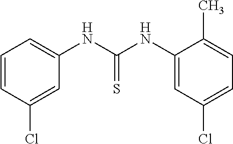 [SMILES: Cc1ccc(Cl)cc1NC(=S)Nc1cccc(Cl)c1] | 1-(5-Chloro-2- methylphenyl)-3-(3- chlorophenyl) thiourea | 40.4 ± 1.2 | 25.8 ± 0.9 | Competitive | 24.6 ± 0.2 |
| 8 | 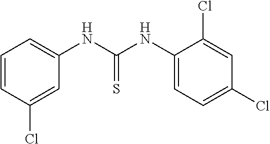 [SMILES: S=C(Nc1cccc(Cl)c1)Nc1ccc(Cl)cc1Cl] | 1-(3-Chlorophenyl)- 3-(2,4- dichlorophenyl) thiourea | 42.4 ± 0.3 | 31.8 ± 3.4 | Mixed | 21.4 ± 0.1 |
| 9 | 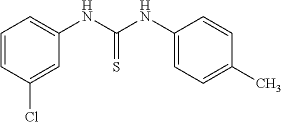 [SMILES: Cc1ccc(NC(=S)Nc2cccc(Cl)c2)cc1] | 1-(3-Chlorophenyl)- 3-p-tolylthiourea | 46.2 ± 0.5 | 39.6 ± 1.0 | Non- Competitive | 2.3 ± 0.4 |
| 10 | 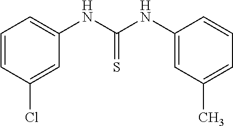 [SMILES: Cc1cccc(NC(=S)Nc2cccc(Cl)c2)c1] | 1-(3-Chlorophenyl)- 3-m-tolylthiourea | 48.5 ± 1.7 | 31.6 ± 1.8 | Mixed | >30 |
| 11 | 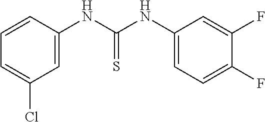 [SMILES: Fc1ccc(NC(=S)Nc2cccc(Cl)c2)cc1F] | 1-(3-Chlorophenyl)- 3-(3,4- difluorophenyl) thiourea | 66.9 ± 4.9 | 49.8 ± 2.6 | Competitive | >30 |
| 12 | 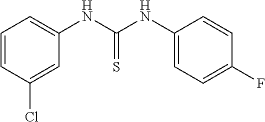 [SMILES: Fc1ccc(NC(=S)Nc2cccc(Cl)c2)cc1] | 1-(3-Chlorophenyl)- 3-(4-fluorophenyl) thiourea | 68.4 ± 8.5 | 62.5 ± 2.5 | Competitive | >30 |
| 13 | 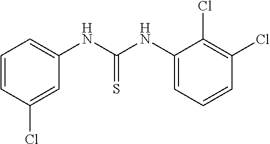 [SMILES: S=C(Nc1cccc(Cl)c1)Nc1cccc(Cl)c1Cl] | 1-(3-chlorophenyl)- 3-(2,3- dichlorophenyl) thiourea | 106.9 ± 9.1 | 87.3 ± 6.7 | Competitive | >30 |
| 14 | 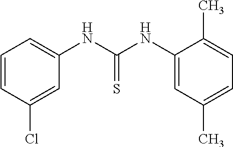 [SMILES: Cc1ccc(C)c(NC(=S)Nc2cccc(Cl)c2)c1] | 1-(3-Chlorophenyl)- 3-(2,5- dimethylphenyl) thiourea | 151.2 ± 8.8 | 106.9 ± 5.5 | Competitive | >30 |
| 15 | 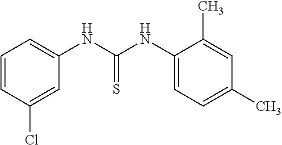 [SMILES: Cc1ccc(NC(=S)Nc2cccc(Cl)c2)c(C)c1] | 1-(3-Chlorophenyl)- 3-(2,4- dimethylphenyl) thiourea | 191.5 ± 9.2 | 172.6 ± 7.9 | Non- Competitive | >30 |
| 16 | 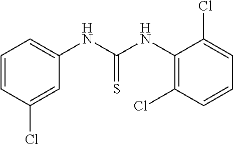 [SMILES: S=C(Nc1cccc(Cl)c1)Nc1c(Cl)cccc1Cl] | 1-(3-Chlorophenyl)- 3-(2,6- dichlorophenyl) thiourea | 247.3 ± 1.7 | 209.8 ± 11.8 | Competitive | >30 |
| 17 | 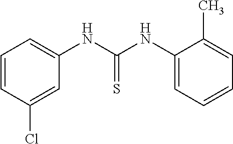 [SMILES: Cc1ccccc1NC(=S)Nc1cccc(Cl)c1] | 1-(3-Chlorophenyl)- 3-o-tolylthiourea | 386.9 ± 13.1 | 329.2 ± 11.6 | Competitive | >30 |
| 18 | 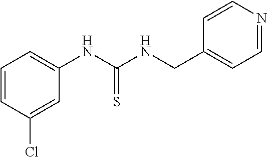 [SMILES: S=C(NCc1ccncc1)Nc1cccc(Cl)c1] | 1-(3-Chlorophenyl)- 3-(pyridin-4- ylmethyl)thiourea | 186.4 ± 12.8 | 148 ± 4.8 | Competitive | >30 |
| 19 | 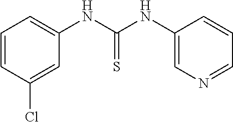 [SMILES: S=C(Nc1cccnc1)Nc1cccc(Cl)c1] | 1-(3-Chlorophenyl)- 3-(pyridin-3-yl) thiourea | 199.3 ± 7.7 | 165.7 ± 5.9 | Competitive | >30 |
| 20 | 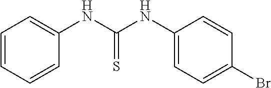 [SMILES: S=C(Nc1ccccc1)Nc1ccc(Br)cc1] | 1-(4-Bromophenyl)- 3-phenylthiourea | 45.7 ± 2.9 | 21.1 ± 2.5 | Competitive | >30 |
| 21 | 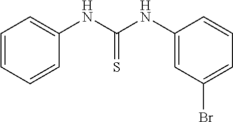 [SMILES: S=C(Nc1ccccc1)Nc1cccc(Br)c1] | 1-(3-Bromophenyl)- 3-phenylthiourea | 79.2 ± 0.6 | 60.8 ± 7.4 | Mixed | >30 |
| 22 | 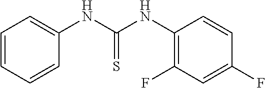 [SMILES: Fc1ccc(NC(=S)Nc2ccccc2)c(F)c1] | 1-(2,4- Difluorophenyl)-3- phenylthiourea | 193.0 ± 1.0 | 142.1 ± 5.0 | Competitive | >30 |
| 23 | 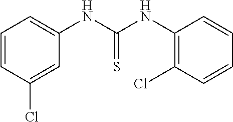 [SMILES: S=C(Nc1cccc(Cl)c1)Nc1ccccc1Cl] | 1-(2- Chlorophenyl)-3- (3-chlorophenyl) thiourea | >500 | NC | NC | NC |
| 24 | 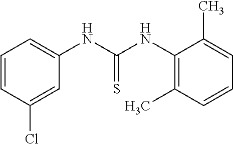 [SMILES: Cc1cccc(C)c1NC(=S)Nc1cccc(Cl)c1] | 1-(3- Chlorophenyl)-3- (2,6- dimethylphenyl) thiourea | >500 | NC | NC | NC |
| 25 | 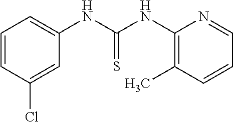 [SMILES: Cc1cccnc1NC(=S)Nc1cccc(Cl)c1] | 1-(3- Chlorophenyl)-3- (3-methylpyridin- 2-yl)thiourea | >500 | NC | NC | NC |
| 26 | 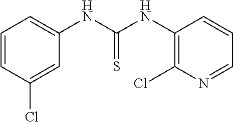 [SMILES: S=C(Nc1cccc(Cl)c1)Nc1cccnc1Cl] | 1-(3- Chlorophenyl)-3- (2-chloropyridin-3- yl)thiourea | >500 | NC | NC | NC |
| 27 | 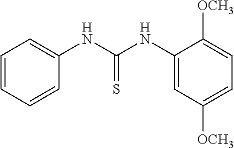 [SMILES: COc1ccc(OC)c(NC(=S)Nc2ccccc2)c1] | 1-(2,5- Dimethoxyphenyl)- 3-phenylthiourea | >500 | NC | NC | NC |
| 28 | 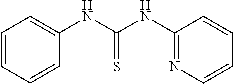 [SMILES: S=C(Nc1ccccc1)Nc1ccccn1] | 1-Phenyl-3- (pyridin-2-yl) thiourea | >500 | NC | NC | NC |
| 29 | 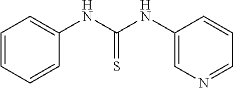 [SMILES: S=C(Nc1ccccc1)Nc1cccnc1] | 1-Phenyl-3- (pyridin-3-yl) thiourea | >500 | NC | NC | NC |
| 30 | 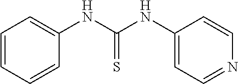 [SMILES: S=C(Nc1ccccc1)Nc1ccncc1] | 1-Phenyl-3- (pyridin-4-yl) thiourea | >500 | NC | NC | NC |
| 31 | 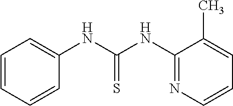 [SMILES: Cc1cccnc1NC(=S)Nc1ccccc1] | 1-(3- Methylpyridin-2- yl)-3- phenylthiourea | >500 | NC | NC | NC |
| 32 | 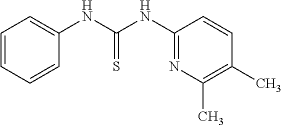 [SMILES: Cc1ccc(NC(=S)Nc2ccccc2)nc1C] | 1-(5,6- Dimethylpyridin-2- yl)-3- phenylthiourea | >500 | NC | NC | NC |
| 33 | 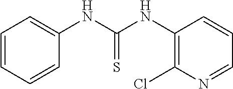 [SMILES: S=C(Nc1ccccc1)Nc1cccnc1Cl] | 1-(2- Chloropyridin-3- yl)-3- phenylthiourea | >500 | NC | NC | NC |
| 34 | 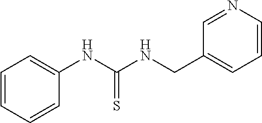 [SMILES: S=C(NCc1cccnc1)Nc1ccccc1] | 1-Phenyl-3- (pyridin-3- ylmethyl)thiourea | >500 | NC | NC | NC |
| 35 | 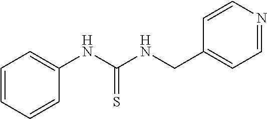 [SMILES: S=C(NCc1ccncc1)Nc1ccccc1] | 1-Phenyl-3- (pyridin-4- ylmethyl)thiourea | >500 | NC | NC | NC |

S.E.M. = Standard Error of Mean at n = 3
NC = Not calculated (because of % of inhibition shown was less than 50% at 500 μM)

1. An inhibitor of alpha chymotrypsin comprising a compound of structure: 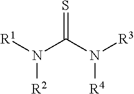
where, R¹=R⁴=H, R²=3-chlorophenyl, and R³=4-chlorophenyl, 3,4-dichlorophenyl, 3-chlorophenyl, 3-methoxyphenyl, 2-methoxyphenyl, 4-methoxyphenyl, 5-chloro-2-methylphenyl, 2,4-dichlorophenyl, p-tolylthiourea, m-tolylthiourea, 3,4-difluorophenyl, 4-fluorophenyl, 2,3-dichlorophenyl, 2,5-dimethylphenyl, 2,4-dimethylphenyl, 2,6-dichlorophenyl, o-tolylthiourea, pyridin-4-ylmethyl, pyridin-3-yl, and
where, R¹=R⁴=H, R²=3-phenylthiourea, and R³=4-bromophenyl, 3-bromophenyl, 2,4-bifluorophenyl.

2. The inhibitor of claim 1, wherein it is used to treat diseases related to proteases, elastases and proteosomes.
